(19) United States
(12) Patent Application Publication
(10) Pub. No.: US 2008/0195075 A1
(43) Pub. Date: Aug. 14, 2008

(54) Adjustable Washable Nappy

(76) Inventor: Rosanna Ruocco, East Sussex (GB)

Correspondence Address: LUEDEKA, NEELY & GRAHAM, P.C., P O BOX 1871, KNOXVILLE, TN, 37901, US

(21) Appl. No.: 11/917,846

(22) PCT Filed: Jun. 22, 2006
(86) PCT No.: PCT/GB2006/002299

§ 371 Date: Dec. 17, 2007

(30) Foreign Application Priority Data
Jun. 23, 2005 (GB) 0512835.0
Jan. 11, 2006 (GB) 0600504.5

(51) Int. Cl. A61F 13/15

(52) U.S. Cl. 604/385.15; 604/385.25; 604/385.01

(57) ABSTRACT

A size adjustable washable nappy (1,201) having a crotch portion which extends between the legs of a wearer and a waistband portion which wraps around the wearer's waist. The nappy (1,201) comprises two side panels (3,5) and a central panel (7) each forming part of the waistband portion and part of the crotch portion. The side panels (3,5) are each provided with releasable attachment means for selective attachment to the other side panel or to the central panel.

The present invention relates to size adjustable washable nappies. In particular the present invention relates to nappies that can be adjusted to adapt to changes in the width between the legs of a child.

Adjustable washable nappies are known in the art. However, it is only possible to adjust these nappies to change the length of the waistband. The prior art nappies are not adjustable to accommodate changes in the distance between a child's hips. As a result, conventional one sized and sized nappies are too big for a new born child. Consequently the fabric of the nappy gathers between the child's legs and is unnecessarily bulky. Also, as a child grows it becomes too large for the nappy and it is frequently necessary for the nappy to be replaced with a larger one. This incurs undesirable expense.

There is therefore a requirement for a nappy that can be adjusted to accommodate increases in the distance between the hips of a child as well as increases in the waist of a child.

Accordingly, the present invention provides a size adjustable washable nappy having a crotch portion extending between the legs of a wearer in use and a waistband portion, the nappy comprising two side panels and a central panel each forming part of the waistband portion and part of the crotch portion, wherein each side panel is provided with releasable attachment means for selective attachment to the other side panel or to the central panel.

In a first embodiment of the nappy according to the present invention the central panel may be formed integrally with the side panels.

In a second embodiment of the nappy according to the present invention the central panel may be formed separately from the side panels.

Preferably the side panels of the nappy of the present invention each comprise first and second waistband portions and a crotch portion therebetween, and means to selectively releasably attach the first waistband portion to the second waistband portion of the same side panel or of the other side panel, in order to secure the nappy in place when wrapped around the wearer.

In one embodiment of the present invention the distance between the two side panels may be changeable by replacement of the central panel.

In an alternative embodiment of the present invention the distance between the two side panels may be changeable by attaching them to the central panel at different positions.

The two side panels may be attachable to the central panel using zip fasteners.

Alternatively, the two side panels may be attachable to the central panel using push fasteners.

In a further alternative embodiment the zip fasteners are arranged such that the side panels may be zipped to the central panel and may also be zipped directly to each other.

In this further alternative one side panel may be provided with half of a zip fastener on the inside surface of the nappy, the other side panel may be provided with half of a zip fastener on the outside surface of the nappy and the central panel may be provided with half of a zip fastener on both its inside and outside surfaces.

In a further alternative embodiment the present invention provides a nappy wherein the two side panels and the central panel may each be provided with a plurality of slots, wherein a set of slots in one of the side panels may be aligned with a set of slots in the central panel, and a set of slots in the other of the side panels may be aligned with a different set of slots in the central panel, and through each pair of aligned sets of slots a tape may be woven in order to attach each side panel to the central panel.

In the preceding embodiments preferably the parts of the first waistband portions are provided with a plurality of rows of fasteners on the surface facing outwardly in use and the second waistband portions are provided with one or more mating fasteners on the surface facing inwardly in use, wherein, the one or more second waistband portion fasteners are selectively attachable to one or more of the first waistband portion fasteners.

Preferably in all embodiments of the nappy the central panel comprises an absorbent pad.

Three preferred embodiments of the present invention will now be described with reference to the accompany drawings, in which:

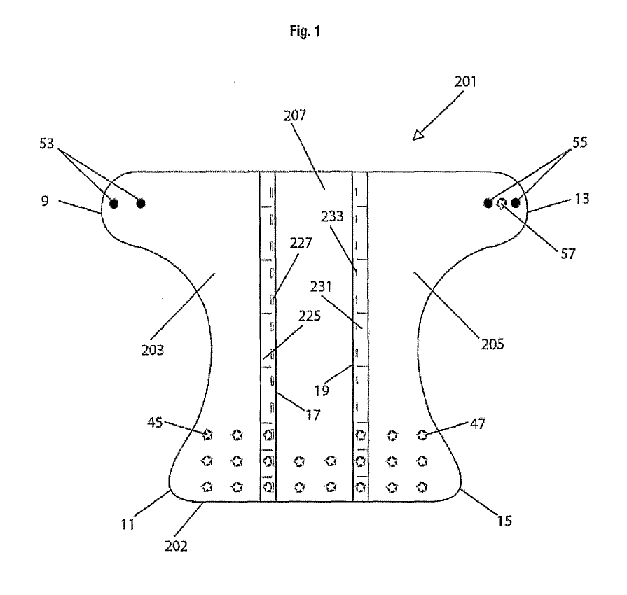

FIG. 1 shows an outside plan view of a first embodiment of a nappy according to the present invention;

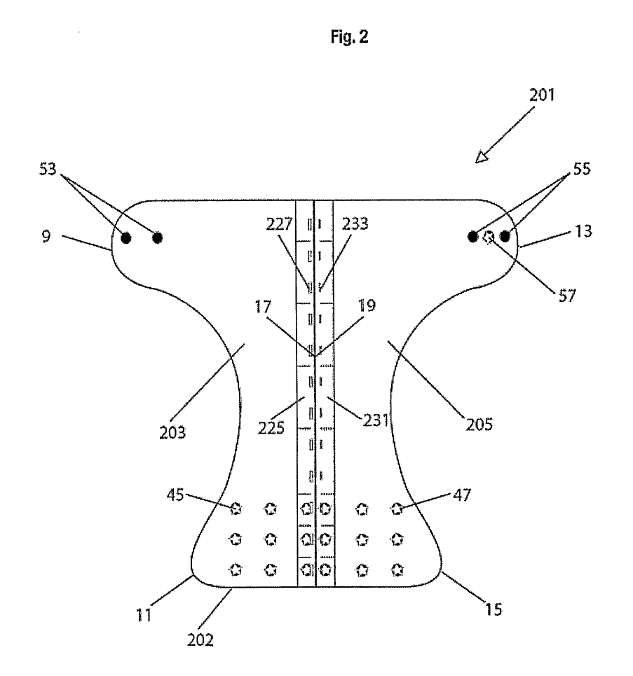

FIG. 2 shows an outside plan view of the first embodiment of a nappy according to the present invention with the side panels attached together;

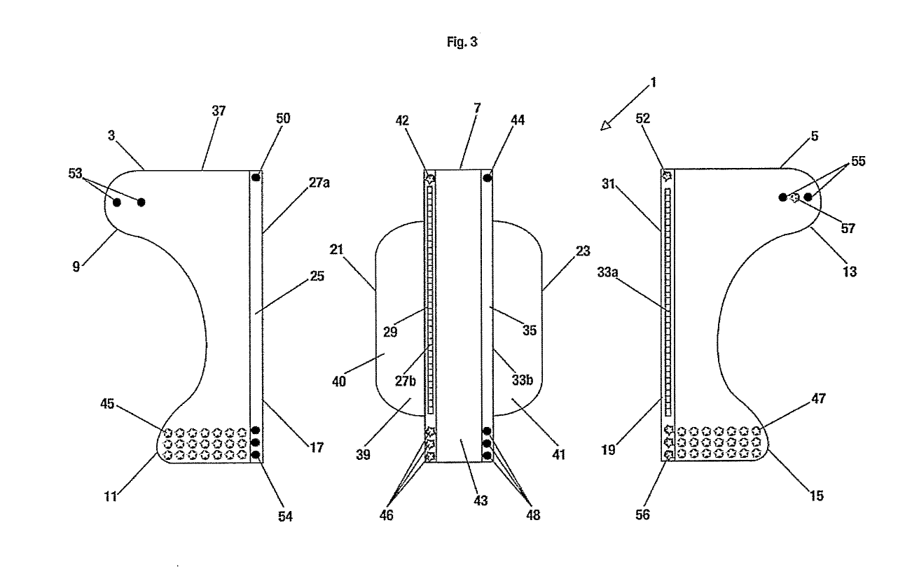

FIG. 3 shows an outside plan view of a second embodiment of a nappy according to the present invention in an unassembled form;

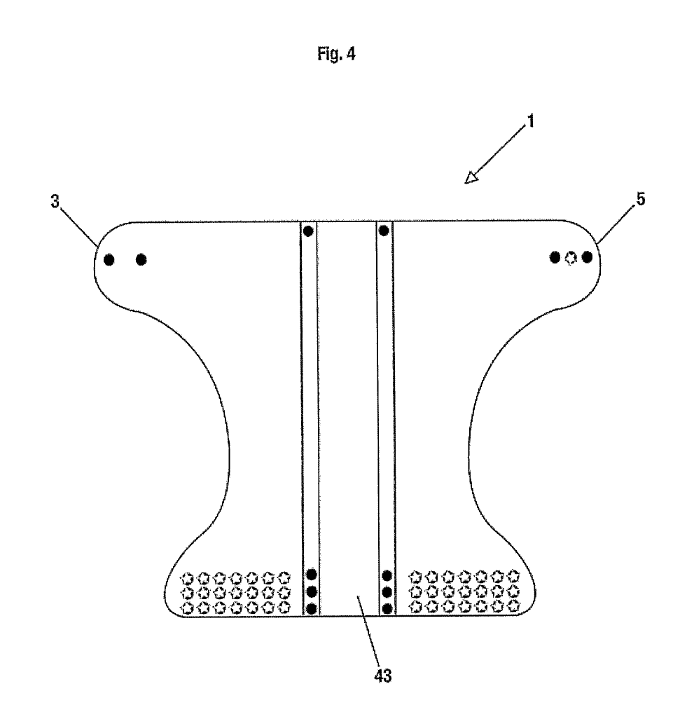

FIG. 4 shows an outside plan view of the second embodiment of a nappy according to the present invention in an assembled form;

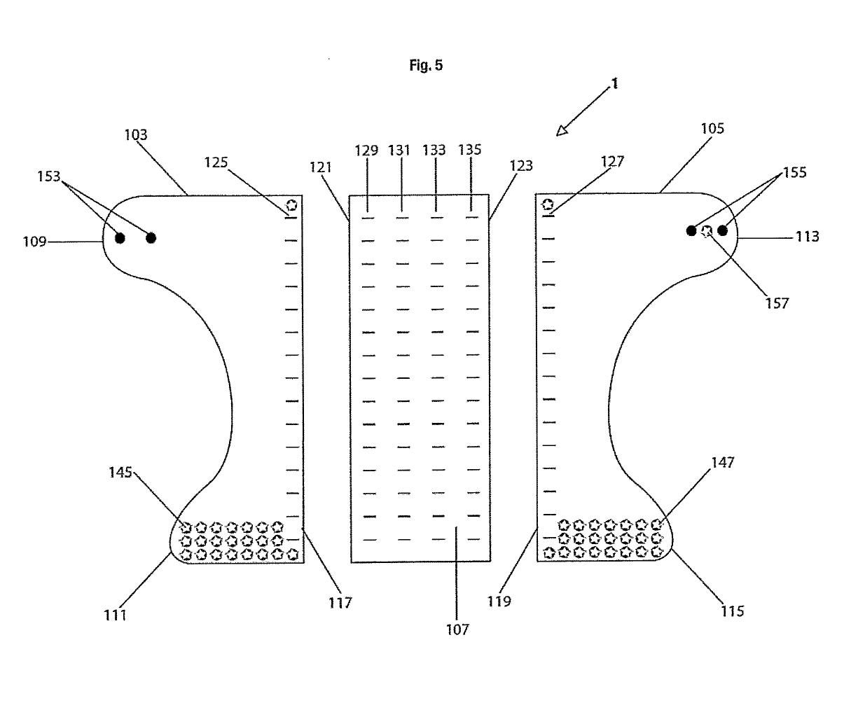

FIG. 5 shows an outside plan view of a third embodiment of a nappy according to the present invention in an unassembled form; and

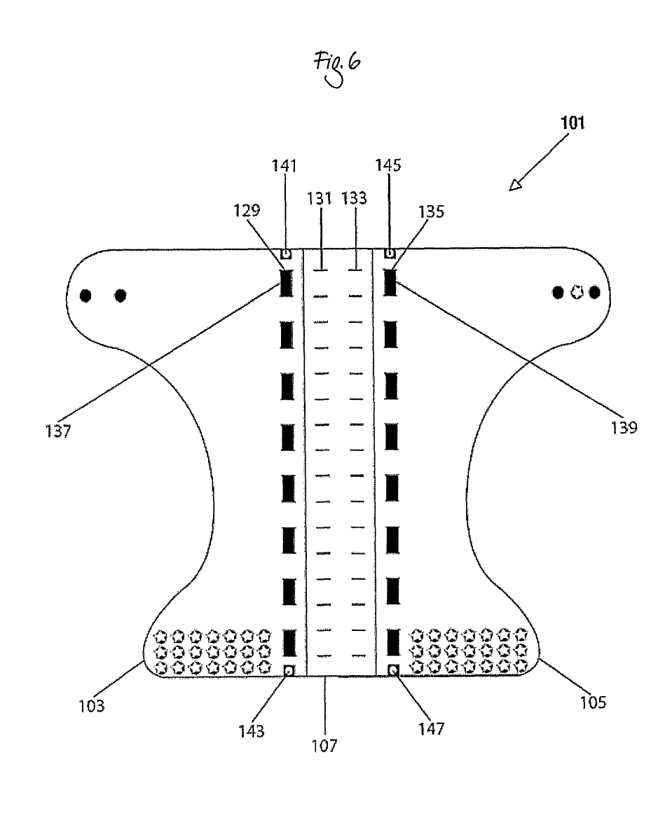

FIG. 6 shows an outside plan view of the third embodiment of a nappy according to the present invention in an assembled form.

A preferred embodiment of a nappy 201 according to the first embodiment of the present invention is illustrated in FIG. 1. The nappy 201 is made up from a piece of material 202, typically a cotton cloth, which is folded and stitched to form two plackets 225,231. These plackets 225,231 divide the nappy 201 into two side panels 203,205 and a central panel 207. This three panel construction permits adjustment of the width of the nappy 201. To reduce the width of the nappy 201 the side panels 203,205 can be attached together. This is explained in further detail below.

A second embodiment of a nappy 1 according to the present invention is illustrated in FIG. 3. The nappy 1 is made up from three separate panels, a first side panel 3, a second side panel 5 and a central panel 7. The side panels 3,5 are attached to opposite edges of the central panel 7. Each of the panels 3,5,7 is made of a cloth material, typically a cotton cloth. This three panel construction permits adjustment of the nappy 1. By replacement of the central panel 7 with a smaller or larger central panel 7, the width of the nappy 1 can be changed so that it fits wearers, for example children, of different sizes.

In the preferred embodiment of a nappy 201 according to the first embodiment of the present invention and in the nappy 1 according to the second embodiment of the present invention each of the side panels 203,205,3,5 respectively takes the form of an elongate rectangle of cloth having two tongues 9,11 and 13,15, respectively, attached at either end of the long edge of the rectangle that is not attached to (in the case of the first embodiment of the present invention) or does not attach to the central panel 207, 7. These tongues 9,11,13,15, in combination with the end sections of the elongate rectangular portion of the side panels 203,205,3,5 and the end sections of the central panel 207,7, form the waistband of the nappy 201,1. The remaining parts of the side panels 203,205,3,5 and the central panel 207,7 form the crotch of the nappy. The tongues 9 and 13 form the end sections of the back half of the waistband and the tongues 11 and 15 form the end sections of the front half of the waistband. The other long edges 17,19 of the elongate rectangular portions of the side panels 3, 5 are each provided with a placket 225,231,25,31 within which is provided respectively one half of a fastener 227,233,27a, 33a. In the first embodiment of the present invention the fastener is a snap fastener, such as the Parallel Tapey fastener from the Tapey Snapper range by Morito. In the second embodiment of the present invention the fastener is a zip fastener. The half of the fastener 227,27a and the half of the fastener 233,33a can be fastened together. In the second embodiment of the present invention at the end of the long edges 17,19 corresponding to the back of the nappy 1 the side panels 3,5 are respectively provided with a male and a female fastener portion 50,52 on the same side as the half of the zip fastener 27a,33a respectively. At the end of the long edges 17,19 corresponding to the front of the nappy 1, the side panels are respectively provided with three male and three female fastener portions 54,56 on the same side as the half of the zip fastener 27a, 33a respectively.

In order to secure the nappy 201,1 to a child the inside surface of the tongue 9 is attached to the outside surface of the tongue 11 and the inside surface of the tongue 13 is attached to the outside surface of the tongue 15. So that the points of attachment of the tongues 9,11,13,15 are at the front of the nappy 201,1 for ease of fitting, the tongues 9 and 13 are longer than the tongues 11 and 15.

In the first embodiment of the nappy according to the present invention the central panel 207, which lies between the plackets 225,231, is rectangular in shape.

In the second embodiment of a nappy according to the present invention the central panel 7 comprises a rectangular transverse section 40, which acts as an absorbent layer on the inside of the nappy 1, and a rectangular and elongate central section 43, that is narrower and longer than the transverse section 40, which forms the body of the nappy 1. The planar sides of the central section 43 and the transverse section 40 are positioned next to each other so that the centre points of each overlay each other and so that the edges of the transverse section 40 are parallel to the edges of the central section 43. The central section 43 is attached to the transverse section 40 by a line of stitching that passes along the centre of the central section 43, parallel to and intermediate its two long edges. As the central section 43 is narrower and longer then the transverse panel 40 the central panel 7 is cross-shaped. The two arms of the cross that are part of the transverse panel 40 form wing sections 39, 41, one on each side of the central section 43, spaced equidistantly from each short edge of the central section 43. The two arms of the cross that are part of the central section 43 respectively form part of the front and back of the waistband of the nappy 1. The wing sections 39, 41 are shorter than the central section 43 so that when the nappy 1 is assembled the wing sections 39, 41 do not overlap the waistband of the nappy 1. This makes the nappy 1 less bulky when the front of the nappy 1 is folded down to decrease its depth. Along each of the long edges of the central section 43 the central panel 7 is provided with a placket 29, 35 within which is provided one half of a zip fastener 27b, 33b. At the end of the long edges of the central section 43, corresponding to the back of the nappy 1, the central section 43 has a female fastener portion 42 adjacent to the same edge and on the same side as the half of the zip fastener 27b and a male fastener portion 44 adjacent to the same edge and on the same side as the half of the zip fastener 33b. At the other end of the long edges, corresponding to the front of the nappy 1, the central section 43 has three female fastener portions 46 adjacent to the same edge and on the same side as the half of the zip fastener 27b and three male fastener portions 48 adjacent to the same edge and on the same side as the half of the zip fastener 33b.

To form the nappy 1 the side panels 3,5 are attached to the central panel 7. To attach the side panels 3,5 to the central panel 7, the halves of the zip fasteners 27a,33a, attached to the side panels 3,5, are zipped together with the halves of the zip fasteners 27b, 33b, attached to the central panel 7, and the fastener portions 42 and 50,44 and 52,46 and 54 and 48 and 56 are attached together. Once the side panels 3,5 have been fitted to the central panel 7 the nappy 1 can be fitted to a wearer, for example a child.

The nappy 201,1 according to the first and second embodiments of the present invention is placed relative to the child such that one of the child's legs is positioned between the tongues 9,11 and the other of the child's legs is positioned between the tongues 13,15. The back tongues 9,13 and the front tongues 11,15 are then brought together around the child's waist. The back tongues 9,13 overlap the front tongues 11,15 on the outside of the nappy 1. The tongues 9,11,13,15 are attached to each other using push fasteners.

To enable the nappy 201,1 to fit the child's waist closely the tongues 9,11,13,15 are provided with a plurality of push fasteners so that the distance around the waistband can be adjusted. The tongues 9,11 are provided on their inside surfaces with male or female fastener portions 53,55. The tongues 11,15 are provided on their outside surfaces with female or male fastener portions 45,47. The tongues 11,15 are provided with three rows of female fastener portions 45, 47, with, for example, seven female fastener portions 45,47 in each row. The male fastener portions 53,55 are fastened into any of the female fastener portions 45,47 contained in the same row in order to form a nappy 201,1 with a waistband of the desired size. However, using these fastener portions 45,47,53,55 the minimum size of the waistband is limited by the tongues 9,13 because when the ends of the tongues 9,11,13,15 meet at the front of the nappy 201,1 the female fastener portions 45,47 are obscured and the waistband of the nappy 1 cannot be made any smaller.

Therefore to form a nappy 201,1 with a smaller waistband the outside surface of one of the tongues 9,13 is provided with a female fastener portion 57. This enables one or more of the male fastener portions 53,55, on one of the tongues 9,13, to engage with one or more of the female fastener portions 45,47 on the opposite side of the nappy 201,1, and one of the male fastener portions 53,55 on the other one of the tongues 9,11 to engage with the male fastener portion 57 on the outside of the first tongue 9,13, when the tongues 9,13 have overlapped.

To enable the nappy 201,1 to fit closely between the child's legs the nappy 201,1 needs to have the correct width. In the first embodiment of a nappy 201 according to the present invention the width of the nappy 201 is changed by connecting the fasteners 227,233 together, to create a smaller nappy, or by disconnecting the fasteners 227,233, to create a larger nappy.

In the second embodiment of a nappy 1 according to the present invention to form a nappy 1 of the correct width the central panel 7 is replaced with a central panel 7 having a different width.

As explained above and as shown in FIG. 3, central panel 7 has three distinct sections. There are two wing sections 39 and 41 and a central section 43. For differently sized central panels 7 the wing sections 39 and 41 remain the same width and the width of the central section 43 changes. To accommodate larger children a central panel 7 having a central section 43 of increased width is used. To accommodate smaller children a central panel 7 having a central section 43 of decreased width is used.

In order to form a nappy 1 with the smallest width the central panel 7 is removed entirely and the side panels 3,5 are zipped directly together. The zip fastener portion 27a, attached to side panel 3, is zipped directly to the zip portion 33a, attached to side panel 5.

It is also possible to adjust the depth of the nappy 201,1, i.e. the distance between the bottom of the nappy 201,1 and the waistband. In order to reduce the depth of the nappy 201,1 the top edges of the tongues 11,15, and the central panel 207,7, are folded inside the nappy 1 so that two, or only one, of the rows of female fastener portions 45,47 are externally visible. The male fastener portions 53,55 on the tongues 9,13 then engage with any of the female fastener portions 45,47 within the externally visible rows.

A third embodiment of a nappy 101 according to the present invention is illustrated in FIGS. 5 and 6. The nappy 101 is made up from three separate panels, a first side panel 103, a second side panel 105 and a central panel 107. The side panels 103,105 are attached to opposite sides of the central panel 107. Each of the panels 103,105,107 is made of a cloth material, typically a cotton cloth.

This three panel construction permits adjustment of the nappy 101, but in a different way to the second embodiment of the present invention. To change the size of the nappy 101, rather than replace the central panel 7 with one of a different size, the side panels 103,105 are instead attached to the central panel 107 at different positions, so that the distance between the side panels 103,105 changes, and hence the width of the nappy 101 changes.

As shown in FIG. 5, each of the side panels 103,105 is substantially the same shape and acts in the same way as the side panels 3 and 5 of the first embodiment.

However, the side panels 103,105 are each provided with a single column of slots 125,127 running adjacent and perpendicular to the long sides 117,119 which are adjacent the central panel 107 when the nappy 101 is assembled.

The central panel 107 is manufactured from a single piece of cloth. The central panel 107 is elongate and rectangular in shape. It is provided with four parallel columns of slots 129,131,133,135 running perpendicular to its long sides 121,123.

To attach the side panels 103,105 to the central panel 107 each of the column of slots 125,127 on the side panels 103,105 is aligned with one of the columns of slots 29,131,133,135 on the central panel 107 and a tape 137,139 is passed through both sets of slots to secure the side panels 103,105 and the central panel 107 together.

To form the largest size nappy 101 the slots 125 on the side panel 103 are aligned with the slots 129 on the central panel 107 and the slots 127 on the side panel 105 are aligned with the slots 135 on the central panel 107, as shown in FIG. 3. The central panel 107 is placed on the inside of the nappy 101. A tape 137 is attached to the central panel 107 by a push fastener 141 attached to the outside surface of the central panel 107. The tape 137 is then threaded through a slot in the side panel 103 and then through an aligned slot in the central panel 107. The tape 137 is then passed back through the central panel 107, through an adjacent slot, and then through an aligned slot in the side panel 103. This process is repeated until the tape 137 exits the final slot in the side panel 103. The tape 137 is then passed over the end of the nappy 101 and attached to the outside surface of the central panel 107 using push fastener 143. This process is repeated to attach the side panel 105 to the central panel 107 using tape 139. Tape 139 is attached to the central panel 107 at one end using push fastener 145 and at the other end by using push fastener 147.

To form a smaller nappy 101 the slots 125 on side panel 103 are aligned with the slots 131 on the central panel 107 and the slots 127 on side panel 105 are aligned with the slots 133 on the central panel 107. The tapes 137 and 139 are woven between the panels 103,105,107 and attached as described above.

For the smallest nappy 101 the central panel 107 is removed and the side panels 103,105 are attached directly to each other by passing a single tape 137,139 through the slots 125,127

Once the side panels 103,105 have been attached to the central panel 107, or have been attached to each other, the nappy 101 can be fitted to a child in the manner described previously in relation to the first embodiment of the present invention.

It will be appreciated that the embodiments of the present invention described above provide a nappy with improved functionality. The skilled reader will realise that a number of variations and modifications to the precise details described herein may be made without departing from the scope of the claims.

1. A size adjustable washable nappy having a crotch portion extending between the legs of a wearer in use and a waistband portion, the nappy comprising two side panels and a central panel, each forming part of the waistband portion and part of the crotch portion, wherein each side panel is provided with a fastener extending along the crotch portion from the back of the nappy to the front of the nappy for selective attachment to the other side panel or to the central panel.

2. A nappy as claimed in claim 1, wherein the central panel is formed integrally with the side panels.

3. A nappy as claimed in claim 1, wherein the central panel is formed separately from the side panels.

4. A nappy as claimed in claim 1, wherein each side panel comprises first and second waistband portions and a crotch portion therebetween, and fastener portions to selectively releasably attach the first waistband portion to the second waistband portion of the same side panel or of the other side panel, in order to secure the nappy in place when wrapped around a wearer.

5. A nappy as claimed in claim 3, wherein the distance between the two side panels is changeable by replacement of the central panel.

6. A nappy as claimed in claim 3, wherein the distance between the two side panels is changeable by attaching them to the central panel at different positions.

7. A nappy as claimed in claim 3, wherein the two side panels are attachable to the central panel using zip fasteners.

8. A nappy as claimed in claim 3, wherein the two side panels are attachable to the central panel using push fasteners.

9. A nappy as claimed in claim 7, wherein the zip fasteners are arranged such that the side panels can be zipped to the central panel and can also be zipped directly to each other.

10. A nappy as claimed in claim 7, wherein one side panel is provided with half of a zip fastener on the inside surface of the nappy, the other side panel is provided with half of a zip fastener on the outside surface of the nappy and the central panel is provided with half of a zip fastener on both its inside and outside surfaces.

11. A nappy as claimed in claim 3, wherein the two side panels and the central panel are each provided with a plurality of slots, wherein a set of slots in one of the side panels is aligned with a set of slots in the central panel, and a set of slots in the other of the side panels is aligned with a different set of slots in the central panel, and through each pair of aligned sets of slots a tape is woven in order to attach each side panel to the central panel.

12. A nappy as claimed in claim 4, wherein the first waistband portions are provided with a plurality of rows of fasteners on the surface facing outwardly in use and the second waistband portions are provided with one or more mating fasteners on the surface facing inwardly in use, wherein, the one or more second waistband portion fasteners are selectively attachable to one or more of the first waistband portion fasteners.

13. A nappy as claimed in claim 1, wherein the central panel comprises an absorbent pad.

14. (canceled)
